(19) United States
(12) Patent Application Publication
(10) Pub. No.: US 2007/0282330 A1
(43) Pub. Date: Dec. 6, 2007

(54) Bipolar high-frequency treatment tool for an endoscope

(75) Inventor: Tomohiro Kawano, Saitama-ken (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/442,423

(22) Filed: May 30, 2006

(51) Int. Cl. A61B 18/14

(52) U.S. Cl. 606/48

(57) ABSTRACT

A bipolar high-frequency treatment tool for an endoscope having a flexible insertion portion configured to be inserted into a body cavity. First and second elongated electrodes mounted to a distal end of the insertion portion so as to be movable between an open position and a closed position. The first and second electrodes being connected with conductive wires that provide a high frequency voltage, and having corresponding first and second surfaces. The first and second surfaces are spaced apart from each other when the first and second electrodes are in an open position, and the first and second electrodes remain spaced from one another when the first and second electrodes are moved to the closed position. In addition, the first and second surfaces may each have generally trapezoidal protrusions, which are configured to prevent tissue damage, extending from respective first and second surfaces to form respective engagement surfaces.

### FIELD OF THE INVENTION

The present invention relates to a bipolar high-frequency treatment tool, and more particularly relates to a tool for an endoscope having first and second surfaces of electrodes pivotally connected that are configured to positively grasp tissue without slipping.

### BACKGROUND AND MATERIAL INFORMATION

It is known in the art to provide a high frequency incision instrument, which utilizes a needle or generally rod-shaped electrode in combination with a large counter electrode placed on the body surface of the patient. High frequency electric current is generated between the needle or rod-shaped electrode and the counter electrode to cauterize the tissue in the vicinity of the needle or rod-shaped electrode. Further, because the high frequency incision instrument, of the rod or needle type, is monopolar there is a large heat impact to a surrounding area of tissue.

The disadvantage of the prior art high frequency incision instrument is that it may make a hole in the tissue that is much deeper than required if the high frequency current is generated continuously for a long period of time since the current flows from one electrode located inside the human body to the other electrode placed on the outer body surface.

Forming such a deep hole can be avoided by generating the current intermittently; however, this requires longer surgery.

Thus, there is a need for a bipolar high-frequency treatment tool for an endoscope that is capable of forming an incision, exfoliation, and hemostasis for performing surgeries, such as a mucosectomy.

### SUMMARY OF THE INVENTION

A non-limiting embodiment of the present invention provides a bipolar high-frequency treatment tool having a flexible insertion portion configured to be inserted into a body cavity through an endoscope. The bipolar high-frequency treatment tool has first and second elongated electrodes mounted to a distal end of an insertion portion so as to be movable between an open position and a closed position. The first and second electrodes are connected to conductive wires that provide a high frequency voltage, and have corresponding first and second surfaces, respectively. The first and second surfaces are spaced apart from each other when the first and second electrodes are in an open position, and the first and second electrodes remain spaced from each other when the first and second electrodes are moved to the closed position. In addition, the first and second surfaces each have spaced apart protrusions which may have any suitable configuration, such as generally trapezoidal protrusions. The protrusions are configured to prevent tissue damage, and in one non-limiting example, extend from the respective first and second surfaces to form respective engagement surfaces.

Another feature includes the first and second electrodes each having inwardly tapered side surfaces that taper toward the first and second surfaces to form narrowed engagement surfaces to increase the current density at the engagement surfaces.

According to another feature, the generally trapezoidal protrusions have a combined length that is shorter in length than the corresponding lengths of the first and second surfaces. In addition, the first and second electrodes are moveable between the open and closed positions along a common plane.

Further, according to another feature, the generally trapezoidal protrusions may have generally rounded corners or edges. In addition, the generally trapezoidal protrusions may be arranged in series to form continuous generally wave-shaped engagement surfaces.

According to another non-limiting embodiment of the present invention, a tool for an endoscope may have a supporting member configured to be connected to an insertion portion of the endoscope. Further, first and second jaws of the tool may be pivotally connected to the supporting member and connectable to operating wires that are operable to move the jaws between open and closed positions. In addition, each jaw may have respective engagement surfaces that remain spaced apart when the first and second jaws are moved to the closed position.

Another feature includes the tool having engagement surfaces having a plurality of protrusions. Further, each of the plurality of protrusions may be provided having a generally trapezoidal shape.

According to another feature of the tool, the first and second surfaces may each have inwardly tapered side surfaces configured to define the engagement surfaces to be narrower in width than the width of the first and second surfaces to increase current density at the engagement surfaces.

According to another feature of the tool, the first and second surfaces each may have inwardly tapered side surfaces configured to define the engagement surfaces to be narrower in width than the width of the first and second surfaces to increase current density at the generally trapezoidal protrusions.

Further, the generally trapezoidal protrusions, provided on the first and second surfaces of respective jaws, may have a combined length that is shorter in length than a respective length of the first and second surfaces. In addition, the generally trapezoidal protrusions may interfit, when in a closed position, to prevent slippage.

According to another feature of the tool, the first and second jaws are moveable between the open and closed positions along a common plane.

According to another feature of the tool, the generally trapezoidal protrusions may have generally rounded corners. Further, the generally trapezoidal protrusions may form continuous generally wave-shaped engagement surfaces.

### BRIEF DESCRIPTION OF THE DRAWINGS

The present invention is further described in the detail description which follows, in reference to the noted plurality of drawings, by way of non-limiting examples of preferred embodiments of the present invention, in which like characters represent like elements throughout the several views of the drawings, and wherein:

FIG. 1 shows a perspective view of a bipolar high-frequency treatment tool in a closed position according to an embodiment of the invention;

FIG. 2 shows a top plan view of the bipolar high-frequency treatment tool of an embodiment of the invention;

FIG. 3 shows a cross-section of the bipolar high-frequency treatment tool taken along line A-A of FIG. 2;

FIG. 4 shows a perspective view of an affected area and the bipolar high-frequency treatment tool in an open position;

FIG. 5 shows a perspective view of the affected area and an incision being formed by the bipolar high-frequency treatment tool when in a closed position;

FIG. 6 shows a perspective view of the bipolar high-frequency treatment tool about to be pressed against a bleeding portion to stop bleeding;

FIG. 7 shows a perspective view of the bipolar high-frequency treatment tool with the electrodes open and positioned to stop bleeding in a wide area; and

FIG. 8 shows a perspective view of the bipolar high-frequency treatment tool pinching a bleeding portion, while current is applied to stop the bleeding.

### DETAILED DESCRIPTION OF THE DRAWINGS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

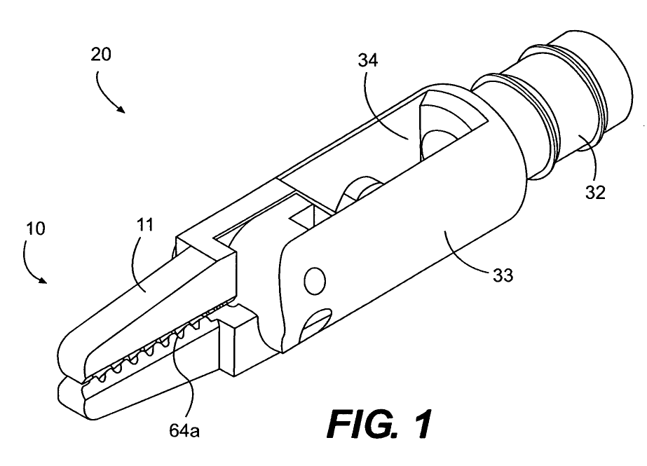

Referring to the drawings, wherein like characters represent like elements, FIG. 1 shows a perspective view of a bipolar high-frequency incision tool 10 for an endoscope according to a non-limiting embodiment of the present invention. The tool may be used in conjunction with a bipolar high-frequency endoscopic surgical system described, for example, in U.S. Pat. No. 6,969,389 and U.S. Patent Publication No. 2003/0191465, both disclosures being expressly incorporated herein by reference in their entireties.

FIGS. 1-3 schematically show a perspective view of a bipolar high-frequency treatment tool 10 for an endoscope according to a first embodiment of the invention. The bipolar high-frequency treatment tool 10 is connectable to a high frequency power supply (not shown).

The bipolar high-frequency treatment tool 10 includes an operation portion (not shown) and an insertion portion 20 connected to a distal end of the operation portion.

The insertion portion 20 is configured to be introduced into a body cavity through a treatment tool insertion channel of an endoscope (not shown). The insertion portion 20 includes an elongated and flexible sheath 30, a pair of conductive wires 31, as shown in FIG. 3, slidably inserted through the sheath 30, and a pair of electrodes 11 (or jaws) provided at the distal end of the insertion portion 20 and connected to the conductive wires 31. The sheath 30 is preferably made of insulating material, e.g., such as poly-tetra-fluoro-ethylene (PTFE).

The conductive wires 31 may be detachable connected to a high frequency power supply (not shown). In this regard, one of the conductive wires 31 is connected to a positive terminal of the power supply and the other to the negative terminal, thereby providing the bipolar high-frequency treatment tool 10.

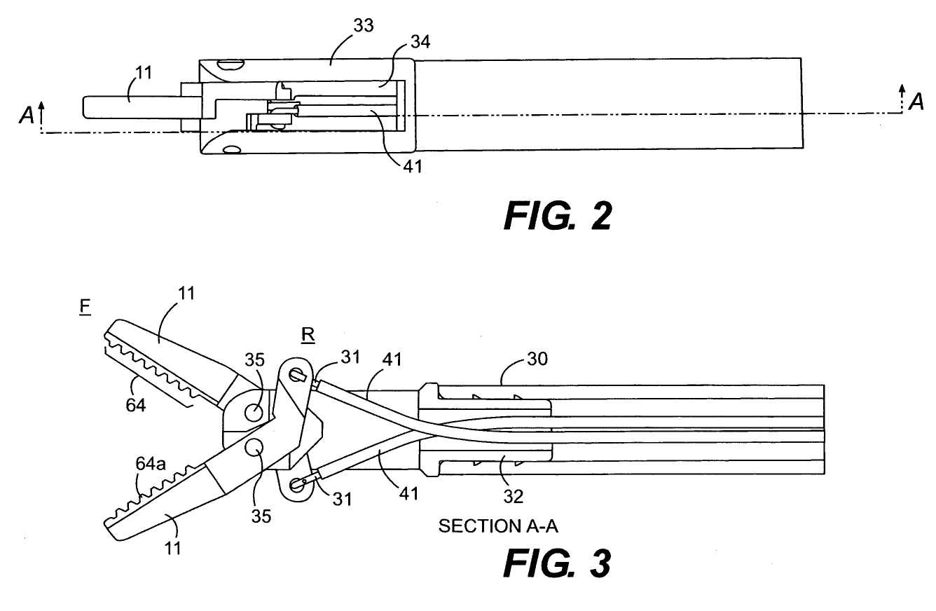

FIG. 3 shows a sectional side view of the distal end portion of the bipolar high-frequency treatment tool 10 shown in FIG. 2. Note that the pair of electrodes 11 is shown in a closed position in FIG. 1, and at an open position in FIG. 3.

As shown in FIGS. 1-3, a supporting member 32 that supports the pair of electrodes 11 is mounted to the distal end of the flexible sheath 30. The supporting member 32 may be made of a hard insulating material, e.g., such as rigid plastic. The supporting member 32 has two arms 33 (FIG. 2) extending in a forward direction and generally parallel to each other to form a slit 34 having a generally uniform width. Two pins 35 are supported between the arms 33 in the vicinity of the distal end thereof. The pins 35 are arranged generally parallel to and spaced apart from each other, and generally perpendicular to side walls of the slit 34. The pins 35 may be made of any suitable material, e.g., stainless steel.

The pair of electrodes 11 are partially provided within the slit 34 of the supporting member 32 and are rotatably mounted to the pair of pins 35. Thus, the pair of electrodes 11 can move between the closed position shown in FIG. 1, at which the electrodes 11 remain spaced from each other, and the open position shown in FIG. 3 in which the electrodes 11 are located further apart from each other.

The rear ends or proximal ends of the electrodes 11 are connected with the conductive wires 31. Each of the conductive wires 31 is covered with an insulating tube 41 except at the end portion thereof, at which the conductive wire 31 is connected to the corresponding electrode 11.

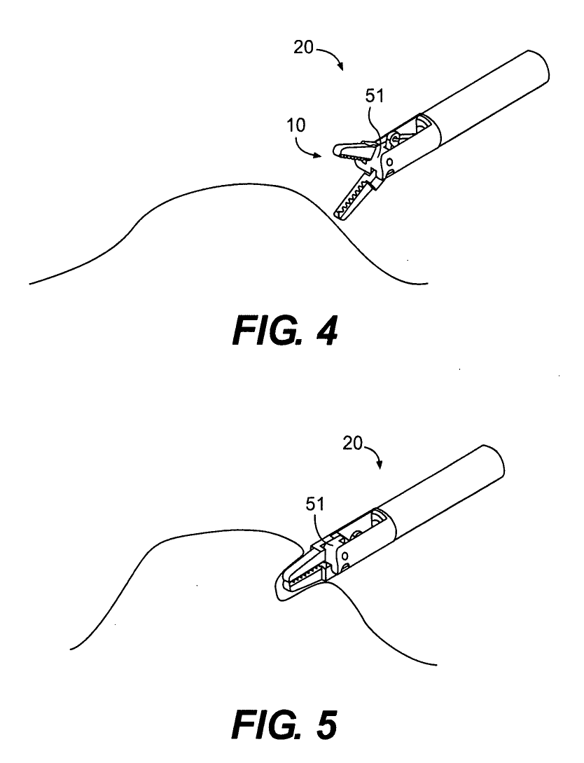

An insulating block 51, as shown in FIGS. 4 and 5, is provided on the outer end of the supporting member 32 to prevent the electrodes 11 from coming into contact with each other within the slit 34. The insulating block 51 is located between the electrodes 11 and may be formed in one piece with the support member 32, or may be formed separately and supported by the pins 35. The insulating block may be made of any suitable insulating material, such as a resin, e.g., poly-tetra-fluoro-ethylene (PTFE).

Referring again to FIG. 3, showing a cross-sectional view of the insertion portion 20, the electrodes 11 are generally elongated opposed members that may be made of any suitable electrode material, such as a metal; e.g., stainless steel. The electrodes 11 include a generally elongated front portion F and a generally elongated rear portion R. When the electrodes 11 are mounted to the supporting member 32, the front portion F is located at a position forward of the arms 33, and the rear portion R is positioned generally between the arms 33 (see FIG. 2).

Two through holes may be provided in the rear portion R of each electrode 11. One through hole, a supporting through hole, is configured to be a supporting hole to receive a pin 35 and is located generally at the center of each electrode 11. The other one is a connection hole for a wire 31 and is provided in the vicinity of the rear portion R of each electrode 11.

As shown in FIG. 2, each electrode 11 is pivotably mounted to the supporting member 32 by insertion the corresponding pins 35 through a respective supporting hole. Thus, each electrode 11 can swing between the closed position shown in FIG. 1 and the opened position shown in FIG. 3.

As can be seen in FIG. 3, the distal end of each conductive wire 31, which is exposed from the corresponding insulating tube 41, is passed through the respective connecting hole to be connected to respective electrodes 11.

The rear portion R of each electrode 11 is slightly bent so that the conductive wires 31 that slide back and forth within the sheath 30 can swing the electrodes 11 around corresponding pin 35 between the open and closed positions.

The electrodes 11 have corresponding first and second surfaces (both labeled 61), respectively, each provided with an engagement surface 64. In one embodiment, the electrodes 11 are configured such that when the electrodes are in the closed position (FIGS. 1, 5, 6 and 8) the engagement surfaces 64 remain slightly spaced, for example by about 0.05 mm to about 0.5 mm, which may result in less tissue damage. In addition, the surfaces 61 each may be provided with inwardly tapered side surfaces 61a configured to form the side surfaces of respective engagement surfaces 64. Further, the narrowed configuration of the engagement surfaces 64, formed due to the tapered side surfaces 61a, increases the current density at the engagement surfaces 64.

Furthermore, the engagement surfaces 64 are preferably formed with protrusions which may have any suitable shape, such as the generally trapezoidal shaped protrusions shown in FIG. 3. Additionally, the generally trapezoidal protrusions 64a (formed on the opposing surfaces 61) may be arranged to interfit to prevent slippage when the electrodes are in a closed position; thereby allowing for a more precise cut. Moreover, the generally trapezoidal protrusions 64a may be provided with rounded corners and edges such that unintentional damage to tissue is prevented.

Furthermore, it should be appreciated that the decrease in surface area at the engagement surfaces 64 (due to the tapered side surfaces of the engagement surfaces increasing the current density), which are configured to contact an affected area, ensures the secure and safe resection of only a desired portion of the affected area.

The engagement surfaces 64 may be provided on the electrodes 11 so that the engagement surfaces 64 are shorter in length than the respective first and second surfaces 61, as shown in FIG. 3.

FIG. 5 shows the engagement surfaces 64 forming an incision in an affected area. In addition, the first and second electrodes 11 are moveable between the open and closed positions along a common plane, shown in FIG. 2. In addition, the generally trapezoidal protrusions 64a, forming the engagement surfaces 64, may be provided having a generally continuous wave-shape (as shown in FIG. 3).

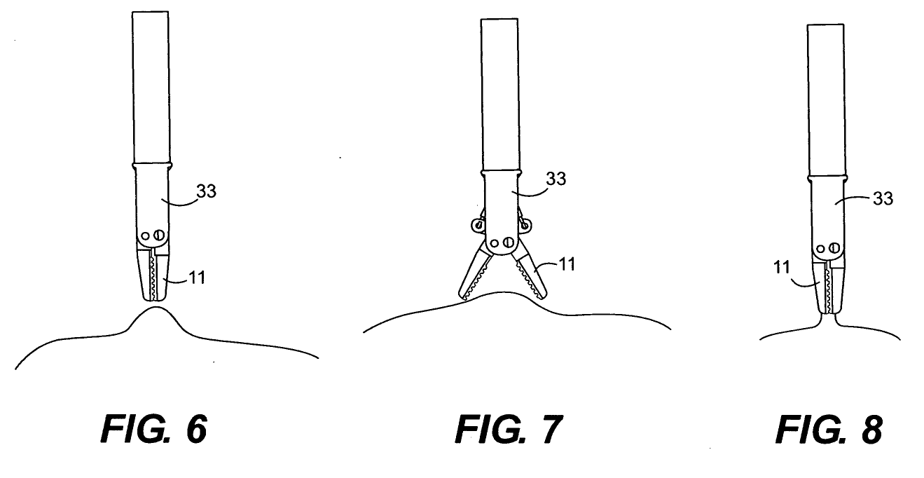

It should also be appreciated that the high-frequency treatment tool 10 can be employed in several ways to provide for a hemostasis operation. In this regard, as shown in FIG. 6, the bipolar high-frequency treatment tool 10 may be pressed against a bleeding portion (while in a closed position) to stop bleeding. Alternatively, when there is a need to stop bleeding in a wide area, the bipolar high-frequency treatment tool 10 may be supplied with current when the electrodes 11 are in an open configuration (see FIG. 7). Further, the bipolar high-frequency treatment tool 10 may pinch the bleeding portion, while current is applied to stop the bleeding, as shown in FIG. 8.

It should be appreciated that a closed end of the bipolar high-frequency treatment tool 10 can be used to mark a desired area of the tissue to be treated. That is, prior to making an incision or performing an hemostasis operation, the surface of the tissue can be marked by applying current to the electrodes while the tool is in the closed position. Thus, the markings allow for a more accurate incision or hemostasis operation.

It is further noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed is:

1. A bipolar high-frequency treatment tool for an endoscope, comprising:
a flexible insertion portion configured to be inserted into a body cavity through an endoscope;
first and second elongated electrodes mounted to a distal end of said insertion portion so as to be movable between an open position and a closed position, said first and second electrodes being connectable to conductive wires that provide a high frequency voltage, said first and second electrodes having corresponding first and second surfaces, respectively;
wherein said first and second surfaces each are provided with a respective engagement surface; and
wherein said engagement surfaces of said first and second surfaces are spaced apart from each other when said first and second electrodes are in an open position, and said engagement surfaces remain spaced apart from each other when said first and second electrodes are moved to said closed position.

2. The bipolar high-frequency treatment tool according to claim 1, wherein said engagement surfaces have a plurality of protrusions extending from respective first and second surfaces.

3. The bipolar high-frequency treatment tool according to claim 1, wherein said plurality of protrusions have a generally trapezoidal shape.

4. The bipolar high-frequency treatment tool according to claim 3, wherein said first and second surfaces each have inwardly tapered side surfaces configured to define said engagement surfaces to be narrower in width than the width of the first and second surfaces to increase current density at the generally trapezoidal protrusions.

5. The bipolar high-frequency treatment tool according to claim 3, wherein said generally trapezoidal protrusions have a combined length that is shorter in length than a respective length of said first and second surfaces.

6. The bipolar high-frequency treatment tool according to claim 3, wherein said generally trapezoidal protrusions interfit, when in a closed position, to prevent slippage.

7. The bipolar high-frequency treatment tool according to claim 1, wherein said first and second electrodes are moveable between said open and closed positions along a common plane.

8. The bipolar high-frequency treatment tool according to claim 3, wherein said generally trapezoidal protrusions have generally rounded corners.

9. The bipolar high-frequency treatment tool according to claim 3, wherein said generally trapezoidal protrusions form continuous generally wave-shaped engagement surfaces.

10. A tool for an endoscope, comprising:
a supporting member configured to be connected to an insertion portion of the endoscope;
first and second jaws pivotally connected to the supporting member and connectable to operating wires that are operable to move the jaws between open and closed positions, said jaws each having an engagement surface; and
wherein said engagement surfaces remain spaced apart when said first and second jaws are moved to the closed position.

11. The tool according to claim 10 wherein each said engagement surface comprises a plurality of protrusions.

12. The tool according to claim 11, wherein each of said plurality of protrusions has a generally trapezoidal shape.

13. The tool according to claim 10, wherein said first and second surfaces each have inwardly tapered side surfaces configured to define said engagement surfaces to be narrower in width than the width of the first and second surfaces to increase current density at the engagement surfaces.

14. The tool according to claim 12, wherein said generally trapezoidal protrusions have a combined length that is shorter in length than a respective length of said first and second surfaces.

15. The tool according to claim 12, wherein said generally trapezoidal protrusions interfit, when in a closed position, to prevent slippage.

16. The tool according to claim 10, wherein said first and second jaws are moveable between said open and closed positions along a common plane.

17. The tool according to claim 12, wherein said generally trapezoidal protrusions have generally rounded corners.

18. The tool according to claim 12, wherein said generally trapezoidal protrusions form continuous generally wave-shaped engagement surfaces.

19. The tool according to claim 12, wherein said first and second surfaces each have inwardly tapered side surfaces configured to define said engagement surfaces to be narrower in width than the width of the first and second surfaces to increase current density at the generally trapezoidal protrusions.
